(19) United States
(12) Patent Application Publication
(10) Pub. No.: US 2023/0293610 A1
(43) Pub. Date: Sep. 21, 2023

(54) COMPOSITIONS AND METHODS FOR TREATMENT OF INFLAMMATION

(71) Applicant: Stephen D Barnhill, League City, TX (US)

(72) Inventor: Stephen D Barnhill, League City, TX (US)

(21) Appl. No.: 18/019,051

(22) PCT Filed: Aug. 1, 2021
(86) PCT No.: PCT/IB2021/057018

§ 371 Date: Jan. 31, 2023

Related U.S. Application Data
(60) Provisional application No. 63/059,937, filed on Jul. 31, 2020.

(51) Int. Cl. A61K 36/06; A61K 31/00; A61K 36/185; A23L 33/105; A61K 45/06

(52) U.S. Cl. CPC A61K 36/06; A61K 31/658; A61K 36/185; A23L 33/105; A61K 45/06

(57) ABSTRACT

Compositions and methods are provided for the treatment of a variety of inflammatory disease processes. The compositions generally comprise at least one edible or medicinal mushroom or extract thereof, at least one cannabinoid, at least one terpene, a curcumoinoid, and at least one flavonoid. Methods for optimizing compositions using artificial intelligence algorithms are also provided.

### CROSS-REFERENCE TO RELATED APPLICATIONS

This claims the benefit of U.S. Provisional Patent Application No. 63/059,937 filed Jul. 31, 2020, the entirety of which is incorporated herein by reference.

### BACKGROUND

### Field of the Invention

This relates generally to compositions and methods useful in the treatment of inflammation and for inflammatory conditions. More particularly this relates to nutraceutical and/or pharmaceutical compositions comprising mushrooms or extracts thereof, cannabis extracts, terpenes, and flavonoids, and the like.

### Description of Related Art

Inflammation is an important natural response to injury, infection, and exposure to deleterious stimuli including pathogens, irritants, cellular damage, and that which is foreign. Inflammation is a complex physiological response mediated by the immune system and without which the body's ability to heal wounds and injuries, fight off infections, and reject, remove, or isolate foreign matter from the body would be severely compromised.

Inflammation results in a predictable set of biological changes, including vasodilation, increased vascular permeability, and extravasation of plasma or other fluids, along with other physical symptoms such as fever, pain, and swelling.

Normal inflammatory responses are controlled, useful, and desirable. However, inflammatory responses can go awry and when they do, the impact on the body can be devastating. Inflammatory responses can become problematic when they are too extreme, when they become chronic, when they are misdirected in terms of location in the body, or when they are triggered by ‘self’ rather than a foreign agent or matter. Chronic inflammation, tissue and organ damages, and auto-immune diseases such as rheumatoid arthritis, systemic lupus erythematosus, gout, inflammatory bowel disease, colitis, psoriasis, and even diabetes can result. While there is much to be learned about the causes and treatment of autoimmune diseases, some autoimmune diseases appear to be connected with T-cell mediated inflammatory responses.

Inflammation is regulated by a range of cellular signals, including for example, cytokines, various secretion by B-cells, cellular responses from T-cells and proinflammatory cells such as macrophages, monocytes, and others, begin producing molecular mediators which initiate and/or maintain the inflammation process. These cellular signals also serve as well-known inflammatory biomarkers including interleukins (e.g. IL-1β, IL-6; IL-8), tumour necrosis factor (TNF-α); nuclear factor-κB (NF-κB), intercellular adhesion molecule-1 (ICAM-1), inducible type cyclooxygenase-(COX-) 2, prostaglandin E2 (PGE2), and 5-lipooxygenase (5-LOX). Another mediator is inducible nitric oxide synthase (iNOS), which is involved in the production of reactive nitrogen species, such as nitric oxide (NO). Many of these biomarkers, as well as others, such as C-reactive protein, can be useful for identifying inflammatory states.

Treatment of inflammation and inflammatory diseases generally involves administering pharmacological agents such as corticosteroids, or a class of drugs known as non-steroidal anti-inflammatory drugs (NSAIDS), both of which are intended to suppress the immune response and decrease the inflammation process.

The risks of such anti-inflammatory drugs are well-known. In the most general sense, all things being equal, any person receiving an immunosuppressive drug is more susceptible to getting an infection. Oral steroids can increase risk of bacterial, viral, or fungal infections, while inhaled steroids can result in thrush (a fungal infection of throat and/or mouth). Even topical steroids can increase the occurrence of skin lesions, acne and the like.

More specifically, corticosteroids, must be used judiciously and are known to have a number of other side effects as well as a number deleterious consequences from long term use. For example, people on steroid therapy frequently can experience glaucoma, edema, elevated blood pressure, and weight gain (particularly around the face, head, and abdomen), in addition to behavioral and/or psychological effects including severe mood swings, rage, confusion, delirium, and even memory lapse. Other undesirable and potentially serious effects include cataracts, hyperglycemia, diabetes, osteoporosis, adrenal gland suppression, muscle loss or weakness, profound fatigue, loss of appetite, nausea, susceptibility to bruising, and slowed wound healing. Examples of corticosteroids include prednisone, cortisone, betamethasone, dexamethasone, hydrocortisone, prednisolone, and methylprednisolone.

NSAIDs on the other hand were developed in part to solve the problems noted for steroid usage. Examples of NSAIDs include aspirin, ibuprofen, naproxen, Diclofenac, indomethacin, celocoxib, tolmetin, ketorolac, and the like. It is now known that these agents too have significant side effects, particularly on the gastrointestinal (GI) tract. The harmful effects include mucosal lesions, bleeding, peptic ulcers, and intestinal perforation. Other side effects of NSAIDs can include acute or chronic renal failure, nephrotic syndrome, hypertension, and cardiovascular toxicity.

Certain mushrooms have long been used for therapeutic purposes. Potentially therapeutic compounds present in mushrooms that might provide benefits for inflammatory disease processes include complex sugars and polysaccharides (substituted or not) (e.g. glucans, glycosides, glycopeptides, and glycoproteins), terpenes and/or terpenoids, sterols, peptides, amino acids, and other small and large molecules. Certain mushrooms are believed to have therapeutic anti-inflammatory properties.

Cannabis spp. have also been used medicinally and the therapeutic use of both psychoactive and nonpsychoactive components of Cannabis is now being acticiely studiend. The endogenous endocannabinoid system and related endocannabinoid biology was originally believed to be primarily directed to neurological and psychiatric. However, cannabinoids, both naturally occurring and exogenous, are increasingly recognized as having role(s) in both inflammation and autoimmune diseases. A role for endocannabinoids in inflammatory processes has been has been suggested by studying exogenous cannabinoids, particularly naturally occurring cannabinoids from Cannabis sativa, as well as synthetic compounds that can interact with CB1 and CB2 receptors. In vitro and in vivo studies have shown that endocannabinoids can help regulate inflammatory processes.

The ongoing discovery of less abundant cannabinoids continues to broaden our knowledge about the range of cannabinoids in plants, and their ability to function in regulation of the endocannabinoid system in humans. Our understanding of the role and potential therapeutic value of such compounds is ongoing. Thus, the use of exogenous cannabinoids and their ability to regulate (particularly upregulate) the endocannabinoid system as a therapeutic approach is being studied.

Other compounds such as terpenes, flavonoids and various botanicals are known to provide beneficial and healthful functions when consumed or administered. Cannabis and mushrooms both have terpenoids and other potentially useful compounds.

There are ongoing concerns about available treatments, side effects, toxicity, and effectiveness of therapeutics for inflammation. And the socioeconomic impact of these conditions is enormous on the personal level for those suffering from chronic inflammation and autoimmune diseases, their families, employers, and society.

There is a need for new treatment compositions and protocols that are useful for treating inflammatory disease processes and which provide significant new features and benefits.

### SUMMARY

In a first of the several aspects of this disclosure, the inventor has discovered that certain pharmaceutical and/or nutraceutical compositions generally comprising combinations of one or more edible or medicinal mushrooms or an extract, fraction, or isolate thereof, one or more cannabinoids, one or more curcuminoids, one or more terpenes, and optionally, one or more flavonoids.

The mushroom or mushroom extract present in the compositions is from any edible or medicinal mushroom species. The edible or medicinal mushroom in various embodiments comprises one or more of Agaricus blazei, A. bisporus, A. subrufescens, Agrocybe aegerita, A. cylindracea, Albatrellus caeruleoporus, Amanita muscaria, Boletus edulis, Cantharellus cibarius, C. tubaeformis, Cordyceps militaris, Cordyceps sphecocephala, C. pruinose, Caripia montagnei, Cyathus africanus, C. hookeri, Daldinia childiae, Dictyophora indusiata (synonym: Phallus indusiatus), Elaphomyces granulatus, Flammulina velutipes, Fomitopsis pinicola, Grifola frondosa, Ganoderma lucidum, Geastrum saccatum, Inonotus obliquus, Lactarius deliciosus, L. rufus, Lentinus edodes, L. polychrous, Lyophyllum decastes, Phellinus linteus, Pholiota nameko, Pleurotus pulmonarius, Pleurotus ostreatus, Poria cocos, Termitomyces albuminosus, Trametes pubescens.

The cannabinoids can be derived from any Cannabis spp. The terpenes and flavonoids can be derived from Cannabis or from any natural or synthetic source. The compositions optionally include other nutraceutical or botanical compounds to provide additional functionality or support.

The inventor has surprisingly discovered that a composition comprising one or more edible or medicinal mushrooms, one or more cannabinoids, one or more curcuminoids, one or more terpenes, and at least one flavonoid with has many benefits for treatment of inflammatory disease processes, and the compositions provide certain useful properties, such as effectiveness including over a long period of time, low toxicity, no serious side effects, and good tolerance on the part of a wide range of subjects.

In another of its several aspects, provided are methods of treating a subject suffering from an inflammatory disease process. The methods generally comprise administering a therapeutically effective dose of a composition to the subject. The compositions generally comprise one or more edible or medicinal mushrooms or an extract, fraction, or isolate thereof, one or more cannabinoids, one or more curcuminoids, one or more terpenes, and optionally, one or more flavonoids. Additional components can also be included, such as S-adenosylmethionine, methylfolate, omega-3 fatty acids, or a B vitamin, vitamin D or a compound providing a biologically-available form thereof. Other components can include fish oil, primrose oil, or an extract of one or more of black cumin, ginger, fenugreek, licorice, coriander, tomato, carrot, sweet potato, broccoli, green tea, rosemary, hazelnut, walnut, wheat germ, or date, or any combination thereof. Garlic extract, alpha lipoic acid (ALA), niacin, and/or nicotinamide may also be useful herein. The methods are generally applicable to any type of inflammation. Generally, the inflammation comprises a common type of inflammatory disease process such as any form of chronic inflammation, asthma, chronic peptic ulcer, tuberculosis, periodontitis, ulcerative colitis and Crohn's disease, sinusitis, active hepatitis, or any kind of autoimmune or autoinflammatory disease process, including but not limited to Addison disease, celiac disease, irritable bowel disease, psoriasis, dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, or Type I diabetes.

The disclosure also provides a plurality of dosing regimens that utilize the compositions, and variations thereof, on various schedules as dictated by the physiological or psychological health of the subject, and the status of the inflammation.

In yet another aspect, methods are provided for optimizing a composition for use in treatment of a subject suffering from an inflammatory disease process. The methods generally employ the use of artificial intelligence algorithms, such as classification algorithms, regression algorithms, clustering algorithms, or a combination thereof.

The methods generally comprise:
a) providing data on the therapeutic effect on the inflammatory disease process of each of:
   i) a plurality of edible or medicinal mushrooms, mushroom extracts, or components thereof;
   ii) a plurality of cannabinoids or combinations thereof;
   iii) a plurality of curcuminoids or combinations thereof;
   iv) a plurality of terpenes or combinations thereof;
   v) a plurality of flavonoids or combinations thereof; and optionally,
   vi) a plurality of combinations of compositions comprising one or more of mushrooms or extracts thereof, cannabinoids, curcuminoids, terpenes, and flavonoids; or
   vi) a plurality of optional ingredients comprising S-adenosylmethionine, methylfolate, omega-3 fatty acids, or a B vitamin or vitamin D or a compound providing a biologically-available form thereof, fish oil, primrose oil, or an extract of one or more of black cumin, ginger, fenugreek, licorice, coriander, tomato, carrot, sweet potato, broccoli, green tea, rosemary, hazelnut, walnut, wheat germ, date, or any combination thereof, garlic extract, alpha lipoic acid (ALA), niacin, and/or nicotinamide;
b) using an artificial intelligence algorithm to analyze the data for the mushrooms or extracts, cannabinoids, curcuminoids, terpenes, and flavonoids; and
c) generating one or more base profiles of compositions optimized for therapeutic treatment of the inflammatory disease process;
d) optionally, using the artificial intelligence algorithm to analyze the data for the combination compositions and the optional ingredients, and
e) generating one or more complete profiles of compositions with and without the optional ingredients.

The inflammatory disease process generally comprises inflammatory disease process comprises Achalasia, Addison's disease, Adult Still's disease, agammaglobulinemia, Alopecia areata, amyloidosis, Ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, colitis, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, gout, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), Hidradenitis Suppurativa (HS) (Acne Inversa), hypogammalglobulinemia, IgA Nephropathy, IgG4-related sclerosing disease, immune thrombocytopenic purpura (ITP), inclusion body myositis (IBM), inflammatory bowel disease, interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, Multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, Pemphigus, peripheral neuropathy, perivenous encephalomyelitis, pernicious anemia (PA), POEMS syndrome, polyarteritis nodosa, polyglandular syndromes type I, II, III, Polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, Pure red cell aplasia (PRCA), Pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, Stiff person syndrome (SPS), subacute bacterial endocarditis (SBE), Susac's syndrome, sympathetic ophthalmia (SO), systemic lupus erythematosus, Takayasu's arteritis, temporal arteritis/Giant cell arteritis, thrombocytopenic purpura (TTP), thyroid eye disease (TED), Tolosa-Hunt syndrome (THS), transverse myelitis, Type 1 diabetes, ulcerative colitis (UC), undifferentiated connective tissue disease (UCTD), uveitis, vasculitis, vitiligo, and Vogt-Koyanagi-Harada Disease. Long term disease processes that are associated with inflammation include asthma, chronic peptic ulcer, tuberculosis, periodontitis, ulcerative colitis, Crohn's disease, sinusitis, active hepatitis, Behçet's disease, Chronic Atypical Neutrophilic Dermatosis With Lipodystrophy and Elevated Temperature (CANDLE), Deficiency of the Interleukin-1 Receptor Antagonist (DIRA), Familial Mediterranean Fever (FMF), Neonatal Onset Multisystem Inflammatory Disease (NOMID), Tumor Necrosis Factor Receptor-Associated Periodic Syndrome (TRAPS), heart disease, Alzheimer's disease, or other autoimmune or autoinflammatory disease.

The data for use in the artificial intelligence algorithm can be obtained from original experiments or literature review.

In a final aspect, this disclosure provides methods for treating inflammatory disease process in a patient in need thereof. The methods generally comprise the step of administering a composition comprising at least one edible or medicinal mushroom or extract thereof, in combination with at least one cannabinoid, at least one curcuminoid, at least one terpene, and at least one flavonoid. The at least one cannabinoid, at least one curcuminoid, at least one terpene, and at least one flavonoid are conveniently administered separately from, sequentially to, or simultaneously with the edible or medicinal mushroom. The at least one edible or medicinal mushroom or extract thereof is also administered separately from, sequentially to, or simultaneously with the cannabinoid, curcuminoid, terpene, and flavonoid.

These and/or further aspects, features, and advantages of the present invention will become apparent to those skilled in the art in view of this disclosure.

### BRIEF DESCRIPTION OF THE DRAWINGS

[symbol]

### DETAILED DESCRIPTION

Provided herein are compositions and methods for treating inflammation inflammatory disease processes. The methods are applicable to acute or chronic inflammation, as well as autoimmune or autoinflammatory diseases in a subject. Surprisingly, the compositions allow a modern practitioner to combine the benefits of certain compounds found in edible and medicinal mushrooms (such as used for centuries in Chinese and other traditional medicine practices, and by e.g. herbalists throughout the world), with the positive benefits of another natural substance, cannabis, also used for centuries. The compositions further utilize certain beneficial terpenes and flavonoids derived from Cannabis and other natural or synthetic sources, and other nutraceutical or pharmaceutical compounds. Used properly, these compositions have little risk, few side effects, and are effective for producing measurable and lasting results in patients suffering from chronic inflammation or any inflammatory disease process.

### Definitions & Abbreviations

Unless expressly defined otherwise, all technical and scientific terms, terms of art, and acronyms used herein have the meanings commonly understood by one of ordinary skill in the art in the field(s) of the invention, or in the field(s) where the term is used. In accordance with this description, the following abbreviations and definitions apply.

The term “inflammation” as used herein includes both acute and chronic inflammation, and any diseases where the causation or symptomology substantially includes inflammation or inflammatory processes. “Inflammatory diseases processes” as used herein include autoimmune diseases and related conditions, such as Achalasia, Addison's disease, Adult Still's disease, agammaglobulinemia, Alopecia areata, amyloidosis, Ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, colitis, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, gout, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), Hidradenitis Suppurativa (HS) (Acne Inversa), hypogammalglobulinemia, IgA Nephropathy, IgG4-related sclerosing disease, immune thrombocytopenic purpura (ITP), inclusion body myositis (IBM), inflammatory bowel disease, interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, Multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, Pemphigus, peripheral neuropathy, perivenous encephalomyelitis, pernicious anemia (PA), POEMS syndrome, polyarteritis nodosa, polyglandular syndromes type I, II, III, Polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, Pure red cell aplasia (PRCA), Pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, Stiff person syndrome (SPS), subacute bacterial endocarditis (SBE), Susac's syndrome, sympathetic ophthalmia (SO), systemic lupus erythematosus, Takayasu's arteritis, temporal arteritis/Giant cell arteritis, thrombocytopenic purpura (TTP), thyroid eye disease (TED), Tolosa-Hunt syndrome (THS), transverse myelitis, Type 1 diabetes, ulcerative colitis (UC), undifferentiated connective tissue disease (UCTD), uveitis, vasculitis, vitiligo, and Vogt-Koyanagi-Harada Disease. Long term disease processes that are associated with inflammation include asthma, chronic peptic ulcer, tuberculosis, periodontitis, ulcerative colitis and Crohn's disease, sinusitis, active hepatitis, and certain inflammatory disease processes. Also included as inflammatory disease processes are autoinflammatory diseases, such as Behçet's disease, Chronic Atypical Neutrophilic Dermatosis With Lipodystrophy and Elevated Temperature (CANDLE), Deficiency of the Interleukin-1 Receptor Antagonist (DIRA), Familial Mediterranean Fever (FMF), Neonatal Onset Multisystem Inflammatory Disease (NOMID), and Tumor Necrosis Factor Receptor-Associated Periodic Syndrome (TRAPS). Diseases that comprise a substantial inflammatory component are included herein, such as heart disease, Alzheimer's disease, and the like, as well as diseases associated with chronic inflammation such as chronic inflammation, asthma, chronic peptic ulcer, tuberculosis, periodontitis, ulcerative colitis and Crohn's disease, sinusitis, and active hepatitis.

As the skilled artisan will appreciate, as used herein the term “edible” does not mean merely capable of being eaten. In that overly broad sense, even poisonous or toxic mushrooms are ‘edible’ however lethal or sickening or the like. In contrast “edible mushrooms” is used herein in the sense of mushrooms that are used traditionally or in modern times as sources of food, nutrients, nutraceuticals, flavors, and the like. Edible mushrooms are neither toxic or poisonous as consumed.

“Medicinal mushrooms” as used herein means any mushroom species that has been used traditionally or in modern times as a source of medicinal or therapeutic benefits, healing properties, and/or healthful compounds. It should be noted that the medicinal mushrooms may not be edible in all embodiments herein, and may in fact be poisonous if eaten, however, one or more extracts therefrom or components extracted or isolated therefrom may be perfectly useful herein.

Notwithstanding the foregoing exception(s), generally, mushrooms that are edible or medicinal may be grouped together, as there may be many crossovers and it may be difficult to clearly distinguish between the two groups. Examples of edible and/or medicinal mushrooms useful herein include mushrooms of the genera Agaricus, Agrocybe, Albatrellus, Amanita, Boletus, Cantharellus, Cordyceps, Caripia, Cyathus, Daldinia, Dictyophora, Elaphomyces, Flammulina, Fomitopsis, Grifola, Ganoderma, Geastrum, Inonotus, Lactarius, Lentinus, Lyophyllum, Phellinus, Pholiota, Pleurotus, Poria, and Termitomyces. Species of particular interest include Agaricus blazei, A. bisporus, A. subrufescens, Agrocybe aegerita, A. cylindracea, Albatrellus caeruleoporus, Amanita muscaria, Boletus edulis, Cantharellus cibarius, C. tubaeformis, Cordyceps militaris, C. pruinose, Caripia montagnei, Cyathus africanus, C. hookeri, Daldinia childiae, Dictyophora indusiata (synonym: Phallus indusiatus), Elaphomyces granulatus, Flammulina velutipes, Fomitopsis pinicola, Grifola frondosa, Ganoderma lucidum, Geastrum saccatum, Inonotus obliquus, Lactarius deliciosus, L. rufus, Lentinus edodes, L. polychrous, Lyophyllum decastes, Phellinus linteus, Pholiota nameko, Pleurotus pulmonarius, Poria cocos, and Termitomyces albuminosus.

More generally edible and/or medicinal mushrooms can be useful in connection with the current disclosure for treating inflammation, directly or indirectly. For example, the therapeutic uses may include: preventing and/or treating inflammatory disease process, helping recover from chemotherapy and/or other treatments with significant toxicity, stimulating and/or supporting the immune system, treating primary or secondary infections, or providing antibacterial or antifungal properties, reducing the side effects of steroidal or nonsteroidal treatment, reducing or mitigating the psychological affects (e.g. stress, anxiety, or the like) of a chronic inflammation or autoimmune diagnosis, generally promoting of health, providing antioxidant functionality, reducing nausea or swelling, stimulating or promoting immune system health, or the like.

Specific anti-inflammatory therapeutic properties include functioning as a reactive oxygen species inducer, a mitotic kinase inhibitor, an anti-mitotic, an angiogenesis inhibitor, a topoisomerase inhibitor, a stimulator of apoptosis, a stimulator of DNA editing and/or repair functions, or as a general immunomodulatory or immunostimulatory compound.

Immune system functions can include stimulating cellular aspect of immunity such as monocytes, natural killer (NK) cells, and dendritic cells. Other potential functions include stimulating T-cell activity, or preventing T-cell apoptosis, stimulating apoptosis of cell that are maintain the inflammatory state.

Reishi mushrooms have been reported to calm the central nervous system and/or have neuroprotective effect, and stimulate aspects of the immune system. Reishi has been reported to have a beneficial effect on the adrenals, and to be anxiolytic, reducing anxiety, and promoting sleep. Reishi has also been associated with improved memory, and sharpened concentration and focus. Chaga mushrooms reportedly have excellent anti-inflammatory properties stemming form certain phenolic content. Cordyceps has been reported to have adaptagenic properties and stimulate the adrenals glands and modulates the nervous system.

“Cannabis” or “Cannabis spp.” as used herein refers to any plant of the genus Cannabis, including plants that may be classified as Cannabis sativa, Cannabis indica, or Cannabis ruderalis. It is well-known that despite the foregoing list, some experts believe that there are only 2 species, and still others consider that there is only a single species (generally, C. sativa). Whatever nomenclature is used, for purposes of this disclosure, “Cannabis” includes all possible members of the genus, without regard to the species to which they are assigned.

As used herein ‘cannabinoids’ means any of a class of compounds that generally can interact with one or more cannabinoid receptors, including the receptors of the endocannabinoid system, in particular, CB1 and CB2. Cannabinoids include e.g., phytocannabinoids and synthetic cannabinoids. Phytocannabinoids are found in several plant species, especially Cannabis spp. Among the most prevalent and most studied cannabinoids are tetrahydrocannabinol (THC), and cannabidiol (CBD). However, there are at least ˜120 known cannabinoids that have been identified in Cannabis within certain classes including the tetrahydrocannabinols, cannabidiols, cannabigerols, cannabinols, cannabichromenes, and cannabinodiol. Other cannabinoids, such as cannabicyclol, cannabieslsoin, cannabitriol are currently classed as ‘miscellaneous’ by some researchers. THC is not only a major cannabinoid in Cannabis spp., it is generally the compound responsible for the psychoactive effects of consuming Cannabis. However, other cannabinoids, such as cannabinol may also be at least mildly psychoactive. Certain other cannabinoids such as CBD may help regulate or attenuate the psychoactive effects of other cannabinoids. For purposes herein, compositions may be created with various ratios of cannabinoids, such as the ratio of CBD to THC or other ratios depending the specific person or the specific condition being treated.

As used herein, “curcuminoids” means any of the compounds associated with turmeric or curcumin, as derived rhizome of the plant Curcuma longa, or synthetic versions or derivative thereof. Curcuminoids include but are not limited to curcumin (aka diferuloylmethane), analogs of curcumin such as demethoxycurcumin (DMC), bisdemethoxycurcumin (BDMC), turmerones, and turmeric oil. Also include are metabolites of curcumin such as tetrahydrocurcumin (THCU), hexahydrocurcumin, and octahydrocurcumin. Conjugates, such as curcumin glucuronide and curcumin sulfate, are also included herein. Conjugation may also provide opportunities for improved delivery of curcumins herein, for example, conjugation to peptide carriers, or polylactic-co-glycolic acid [PLGA]; as well as complexation with essential oils; coadministration with piperine; and encapsulation into nanoparticles, liposomes, phytosomes, polymeric micelles, and cyclodextrins may also be useful herein.

As used herein, “terpenes” means any of the organic compounds commonly known as terpenes or terpenoids. Terpenes are generally aromatic compounds classified as isoprene derivatives. Terpenes suitable for use herein include hemiterpenes, monoterpenes, sesquiterpenes, diterpenes, sesterterpenes, triterpenes, sesquaterpenes, tetraterpenes, polyterpenes, and norisoprenoids. Exemplary terpenes that are particularly useful herein include alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, and valencene.

As used herein, “flavonoids” includes any of the class of polyphenolic molecules containing 15 carbon atoms that are naturally produced in plants and are soluble in water. Also included herein as “flavonoids” are natural or synthetic derivative or analogs thereof that have biological activity. Flavonoids of use herein can generally be divided in to 6 groups of structurally-related compounds: chalcones, flavones, isoflavonoids, flavanones, anthoxanthins and anthocyanins. Also useful are flavanols and catechins, as well as glucosides or other derivatives or analogs of any of the foregoing. The flavonoids are found in most fruits and vegetables, particular colorful ones. They are also prevalent in legumes (including soybeans), grains, green and black teas, as well as red wine.

Flavonoids have numerous functions in plants, and act as important cell messengers. Various flavonoids are believed to provide healthful benefits and functions to humans such as anti-viral, anti-cancer, anti-inflammatory, anti-allergic, and anti-oxidant properties. They may also be cardio-protective, cholesterol-lowering, and anti-atherosclerotic. Natural or synthetic flavonoids from any source may be used herein. Generally natural flavonoids are preferred. Flavonoids isolated from Cannabis, such as cannaflavins A, B, and or C, are of interest in certain applications, as are vitexin, isovitexin, apigenin, kaempferol, quercetin, orientin, and luteolin, as well as the catechins found in Cannabis.

“Traditional Jamaican medicinal plants” means any plant that has been used in traditional or indigenous medicine or herbalism practices in Jamaica or other Caribbean states. The book, “Common Medicinal Plants of Portland, Jamaica” by Thomas and Austin, provides a useful list of a number of such plants. The book was published in its second edition in 2010 by CIEER. For purposes herein, the definition of such traditional Jamaican medicinal plants” expressly excludes Cannabis spp.

As used herein, the singular form of a word includes the plural, and vice versa, unless the context clearly dictates otherwise. Thus, the references “a”, “an”, and “the” are generally inclusive of the plurals of the respective terms. For example, reference to “a composition” or “a mushroom extract” includes a plurality of such “compositions” or “mushroom extracts.”

The words “comprise”, “comprises”, and “comprising” are to be interpreted inclusively rather than exclusively. Likewise, the terms “include”, “including” and “or” should all be construed to be inclusive, unless such a construction is clearly prohibited from the context. Further, forms of the terms “comprising” or “including” are intended to include embodiments encompassed by the phrases “consisting essentially of” and “consisting of”. Similarly, the phrase “consisting essentially of” is intended to include embodiments encompassed by the phrase “consisting of”.

Where used herein, ranges are provided in shorthand, so as to avoid having to list and describe each and every value within the range. Any appropriate value within the range can be selected, where appropriate, as the upper value, lower value, or the terminus of the range.

The methods and devices and/or other advances disclosed here are not limited to particular methodology, protocols, and/or structures described herein because, as the skilled artisan will appreciate, they may vary. Further, the terminology used herein is for the purpose of describing particular embodiments only, and is not intended to, and does not, limit the scope of that which is disclosed or claimed.

Although any devices, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent permitted under applicable law. Any discussion of those references is intended merely to summarize the assertions made therein. No admission is made that any such patents, patent applications, publications or references are prior art, or that any portion thereof is either relevant or material to the patentability of what is claimed herein. Applicant specifically reserves the right to challenge the accuracy and pertinence of any assertion that such patents, patent applications, publications, and other references are prior art, or are relevant, and/or material.

### Abbreviations

The following abbreviations apply unless indicated otherwise:

APM: “Apollon Medical” strain of C. sativa;

BDMC; bisdemethoxycurcumin;

CBD: cannabidiol;

CBG: cannabigerol;

CBN: cannabinol;

DMC: demethoxycurcumin;

DMT: N,N-Dimethyltryptamine;

5-HT: 5-hydroxytryptamine;

KNN: K Nearest Neighbor;

NK: natural killer cells;

NSAID: nonsteroidal anti-inflammatory drugs;

OCD: obsessive compulsive disorder;

PKCO: protein kinase C theta;

PLGA: polylactic-co-glycolic acid;

SVM: Support Vector Machines;

THC: tetrahydrocannabinol; and

THCU: tetrahydrocurcumin.

### DETAILED DESCRIPTION OF ILLUSTRATIVE EMBODIMENTS

In a first of its several aspects, compositions generally comprising one or more edible or medicinal mushrooms or extracts, fractions, isolates, or components thereof are provided. The compositions further generally comprise one or more cannabinoids, one or more terpenes, and optionally, one or more flavonoids.

In various embodiments, the edible or medicinal mushroom(s) comprise one or more of Agaricus, Agrocybe, Albatrellus, Amanita, Boletus, Cantharellus, Cordyceps, Caripia, Cyathus, Daldinia, Dictyophora, Elaphomyces, Flammulina, Fomitopsis, Grifola, Ganoderma, Geastrum, Inonotus, Lactarius, Lentinus, Lyophyllum, Phellinus, Pholiota, Pleurotus, Poria, and Termitomyces.

These mushrooms are all well-known edible and/or medicinal mushrooms with a long history of use. Without limiting the invention to any particular theory of operation, mushrooms, such as Reishi, Chaga, shitake, maitake, turkey tail, and many others have a plethora of beneficial and potentially therapeutic compounds present in them. For example, the polysaccharides, peptides, and/or other small molecules in various mushroom have been shown to regulate the production of certain molecular mediators of inflammation and functions of immune cells (e.g. B- and/or T-cells) as well as pro-inflammatory lymphocytes, T-helper cells, T-killer cells, and macrophages. The same glycan has also been shown to be involved with suppressing tumor necrosis factor (TNF-α). Phenolics like botulin and betulinic acid in Chaga are believed to have anti-inflammatory properties.

Regardless of the mechanism(s) responsible, the compositions preferably comprise the benefits of such edible or medicinal mushroom(s) as described above.

The mushroom(s) in certain presently preferred embodiments comprises one or more species such as Agaricus blazei, Albatrellus confluens, Antrodia camphorate, Boletus badius, Clitocybe maxima, Cordyceps militaris, Cordyceps sinensis, Cordyceps liangshanensis, Cordyceps gunnii, Cordyceps cicadicola, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, or Trametes versicolor.

The edible or medicinal mushrooms or extracts can be present as fresh mushrooms, or as a dried mushroom or extract thereof, a lyophilized mushroom preparation, a mushroom powder, or an aqueous or alcohol extract (e.g. an ethanolic or other alcohol extract) of mushroom. Extracts for purposes herein can include hot- or cold-water extracts. Mushroom concentrates, or partially purified or even extensively purified mushroom fractions, or fully purified components or isolates from a mushroom are also useful herein.

In certain embodiments, preferably, various extracts, concentrates, or partially purified fractions, or the like, are enriched for one or more advantageous mushroom component, or compounds with desirable effects on treatment of inflammation, immune status, or general health. In one embodiment, the extract, concentrate, partially purified fraction, or the like are enriched for one or more phenolic acids, flavonoids, triterpenes, carotenoids, stilbenes, sterols, fatty acids, lignans, chitosan, polyphenols, polysaccharides, glycosides, glucans, polysaccharide-protein complexes, glycoproteins, polysaccharopeptide, krestin, tocopherols, peptides, cyclic peptides, or other amino compounds found in the mushroom.

While certain edible and/or medicinal mushrooms have been explored for their use in the therapeutic treatment of inflammation, the inventors have discovered that by providing mushroom extracts with additional compounds such as cannabinoids, terpenes, flavonoids, and other components, antiinflammation benefits can be derived from the treatments with better outcomes and fewer side effects.

The compositions in various embodiments therefor also include cannabinoids, comprising one or more of cannabidiol (CBD), cannabinol (CBN), cannabigerol (CBG), or tetrahydrocannabinol (THC).

In certain embodiments, the cannabinoids are derived from hemp, or the THC content is removed from the cannabinoids such that THC is present in the composition at less than about 0.3 percent. In various embodiments, the THC content may be less than 0.2, or even less than 0.1% of the composition.

In other embodiments, the ratio of cannabinoids is set in the compositions. For example, in certain embodiments, the ratio of CBD to THC ranges from about 1:5 to about 30:1 or more. In other embodiments the ration may range from about 1:2 to about 10:1. In still other embodiments, the ratio of CBD to THC may be about 1:1 to 5:1. In other cases, the ration of e.g. CBD to CBG or CBN may be set based on any of a variety of factors, including the health status of the subject being treated, the symptoms of the subject, the condition(s) being treated, and or one or more physiological or genetic criteria.

In certain embodiments the compositions may be provided in completely customized or personalized formulations for each person being treated—i.e. as personalized medicines. In such cases, the compositions may be adjusted based on initial or subsequent blood work, enzyme test results, bioinformatic data (including measurements of e.g. the genome, transcriptome, proteome, metabolome, or any portion thereof, for a subject), the nature of the inflammation, specific markers, antigen, or mediators of the inflammatory disease process, specific symptomology, or the like. The formulation of the compositions may also be changed based on the results from an initial treatment, subsequent treatment, or based on subsequent tests.

In various presently preferred embodiments, the cannabinoids are derived from Cannabis spp. or an extract thereof. Cannabis can generally be concentrated or extracted (e.g. via mechanical or chemical means) to obtain cannabinoids. Mechanical means of extracting oils from plants, such as pressing, have been used for centuries, and may be suitable for use herein. Extraction via chemical means includes extraction with various volatile solvents that range from hydrocarbon solvents such as butane, hexanes or propane, to supercritical fluids, alcohol (e.g. isopropanol, butanol, or ethanol), steam, or even water. Two very common methods are extraction with supercritical carbon dioxide, or ethanol, both of which are particularly useful herein. Extracts can be also be distilled e.g. to remove additional compounds of interest, or to concentrate them. Certain components can be removed, e.g. by treatment with steam to strip certain volatiles, which can be captured as an additional component from the Cannabis.

In various embodiments, cannabinoids present in an ethanolic extract or supercritical CO₂ extract of Cannabis sativa are preferred for use herein. The extract comprises one or more of Ringo's Gift, Harle Tsu, ACDC, Charlotte's Web, The Gift, or Pineberry strains of Cannabis sativa. “Apollon Medical” (“APM”), a proprietary strain commercially available from Apollon Formularies, is also useful herein.

In one embodiment, the curcuminoids in the composition comprise one or more of curcumin, demethoxycurcumin (DMC), bisdemethoxycurcumin (BDMC), turmerones, turmeric oil, or a natural or synthetic metabolites, conjugates, or derivatives thereof. In certain preferred embodiments, the curcuminoids presents are derived from rhizomes of Curcuma longa. They may be for example, in the form of crude extract or they be in an enriched fractions, semi, or partially purified, or they be substantially purified compounds.

The compositions in various embodiments include terpenes comprising one or more monoterpenes, one or more sesquiterpenes, or a combination thereof. The terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene.

In one embodiment, the terpenes comprise phytol, limonene, humulene, myrcene, phellandrene, caryophyllene, linalool, pinene, or a combination thereof. In other embodiments, the terpenes preferably comprise one or more of limonene, myrcene, beta-caryophyllene, linalool, alpha pinene, or a combination thereof.

In presently preferred embodiments, the terpenes are derived from Cannabis spp. or an extract thereof. The terpenes can be derived from any source and in certain embodiments, they can be present in steam distillate or an ethanolic extract of Cannabis sativa.

The compositions in certain embodiments include extracts of Cannabis spp, such as C. sativa as a source of cannabinoids and/or terpenes. In various embodiments, the C. sativa comprises one or more of Ringo's Gift, Harle Tsu, ACDC, Charlotte's Web, The Gift, or Pineberry strains. In other embodiments, the C. sativa comprises the proprietary Apollon Formularies strain, APM.

In various other embodiments, the compositions may further comprise one or more flavonoids. The flavonoids can comprise chalcones, flavones, isoflavonoids, flavanones, anthoxanthins, anthocyanins, flavonols, or glucosides or other biologically active derivatives or analogs thereof, and combinations of any number of the foregoing. In certain embodiments, flavonoids are included in the compositions are isolated or derived from a plant, mushroom, or other natural source.

In another embodiment, the composition still further comprises an extract or fraction from one or more traditional Jamaican medicinal plants other than Cannabis spp. Any of the traditional Jamaican or Carribbean medicinal plants may be useful herein. Examples of Jamaican plants useful herein includes ackee, aloe vera, avocado, breadflower, ginger, leaf of life, soursop, tamarind, and trumpet. In a presently preferred embodiments, the compositions include a whole plant extract, or an extract from any parts or portion thereof including but not limited to leaves (dried, fresh, or extracted), stems, flowers, roots, fruit, seeds, or the like.

In terms of compounding the compositions, the skilled artisan will appreciate that methods of maximizing the efficacy of the composition such as by enhancing the bioavailability of one or more components, or by providing the components in optimized ratios, for example one component to another with which it interacts, or each component to the others in ratio(s) that optimize the absorption into the gut or bloodstream, or enhance the therapeutic effect of the composition. The skilled artisan will also understand that some information useful in improving the compounding may be obtained empirically.

In various embodiments, the compositions, or one or more components thereof, may be solubilized, micronized, provided as, for example. extracts, powders, lyophilized powders, concentrates, tinctures, essential oils, aqueous or lipid suspensions, emulsions, microemulsions, or nano-emulsions, or in whole or part as liposomal, vesicular, or other delivery systems. As described below, the compounding or formulation of any of the compositions provided herein may be optimized for the intended delivery route.

The compositions may be administered and delivered as pharmaceuticals, however, it is also contemplated that one or more of the compositions may be formulated for administration and delivery by oral routes that include as food and beverages, including solid, semisolid, and liquid foods, such as smoothies, shakes, pudding, broths, teas, and soups. The food and or beverage compositions can also include hot, cold, or even frozen foods (such as frozen desserts).

In another aspect of the disclosure, provided are methods of treating a subject suffering from inflammation. The methods generally comprise administering a therapeutically effective dose of a composition to the subject. The composition typically comprises one or more edible or medicinal mushrooms or an extract, fraction, or isolate thereof, one or more cannabinoids, one or more terpenes, and optionally, one or more flavonoids.

The inflammation in various embodiments the inflammatory disease process comprises any form of chronic inflammation, asthma, chronic peptic ulcer, tuberculosis, periodontitis, ulcerative colitis and Crohn's disease, sinusitis, active hepatitis, or any kind of autoimmune or autoinflammatory disease process, including but not limited to Addison disease, celiac disease, irritable bowel disease, psoriasis, dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, or Type I diabetes.

The composition for use in the methods is generally as described above for the first aspect. The description of the compositions above is incorporated by reference herein for purposes of the present methods.

In various embodiments, the compositions for use in the methods further comprise one or more optional ingredients comprising S-adenosylmethionine, methylfolate, omega-3 fatty acids, or a B vitamin, vitamin D or a compound providing a biologically-available form thereof. In still other embodiments fish oil, primrose oil, or an extract of one or more of black cumin, ginger, fenugreek, licorice, coriander, tomato, carrot, sweet potato, broccoli, green tea, rosemary, hazelnut, walnut, wheat germ, or date, or any combination thereof can also be included. Garlic extract, alpha lipoic acid (ALA), niacin, and/or nicotinamide are also contemplated for use herein.

As above the curcuminoids can comprise one or more of curcumin, demethoxycurcumin (DMC), bisdemethoxycurcumin (BDMC), turmerones, turmeric oil, or a natural or synthetic metabolites, conjugates, or derivatives thereof. In certain presently preferred embodiments, the curcuminoids presents are derived from rhizomes of Curcuma longa. They may be in the form of crude extract or they be in an enriched fractions, semi, or partially purified, or they be substantially purified compounds.

The terpenes for use with the compositions can comprise one or more monoterpenes, one or more sesquiterpenes, or a combination thereof. In various embodiments, the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene. In certain preferred embodiments, the terpenes are derived from Cannabis spp. or an extract thereof. The terpenes can be present in steam distillate or an ethanolic extract of Cannabis sativa, and can comprise limonene, myrcene, beta-caryophyllene, linalool, alpha pinene, or a combination thereof, in some embodiments. The Cannabis sativa in one embodiment includes one or more of Ringo's Gift, Harle Tsu, ACDC, Charlotte's Web, The Gift, or Pineberry strains.

The compositions for use with the methods also comprise one or more flavonoids that can be chalcones, flavones, isoflavonoids, flavanones, anthoxanthins, anthocyanins, flavonols, or glucosides or other biologically active derivatives or analogs thereof, and preferably they are from a plant, mushroom, or other natural source.

Presently preferred flavonoids include cannaflavin A, cannaflavin B, or cannaflavin C, vitexin, isovitexin, apigenin, kaempferol, quercetin, orientin, luteolin, a catechin found in Cannabis, or a combination of any of the foregoing.

In certain embodiments, the methods further comprise one or more steps of administering an additional therapeutically effective dose of the composition.

Preferably the additional administering steps are performed on a periodic basis of any frequency or schedule. For example, the administration or dosing can conveniently be on e.g. a daily, thrice weekly, twice weekly, weekly, biweekly, monthly, bimonthly, quarterly, semi-annual, or annual basis. The administration need not be the same over every period of time. By way of nonlimiting example, administration could be daily for a week, then weekly for a month. Or the administration could be every 4 months for a year, then every 6 months thereafter. Similarly, the actual amount of the composition or dosage administration can vary. For example, a monthly dosage schedule could feature a dose of x for the first dosage each quarter, and a dose of 0.1× for the remaining months in each quarter.

Just as the composition can be ‘personalized’, so can the administration or dosing schedule. Thus, in various embodiments, the methods further comprise the step of periodically assessing one or more of the subject's medication levels, enzyme levels, or other indicators of physiological health or status, genetic markers or metabolic markers or mediator presence, or the like, in order to determine the periodic basis for administration.

The methods provide for administration of the compositions via any useful route, including parenteral (intravenous, intra-arterial, intramuscular, intraperitoneal, or subcutaneous), oral, nasal, ocular, transmucosal (buccal, vaginal, or rectal), transdermal, or via inhalation.

It should be noted that the route of dosing or administration of compositions can vary over the course of treating a subject or patient with multiple steps of treatment, as well as from subject to subject, or with different types of inflammatory disease. For example, administration via one route may be useful when administering a larger dose and a different route may be useful for smaller doses. Or, administration via a particular route may be appropriate initially, with subsequent doses conveniently administered through another route.

In one embodiment, the method further comprises a step of providing to the subject additional treatment of the inflammatory disease comprising:
i) one or more doses of an NSAID;
ii) one or more treatments with coricosteroids;
iii) one or more other treatments specifically provided to treat the inflammatory disease process; or
iv) any combination of any of the foregoing.

Methods that further comprise a step of providing to the subject an additional composition are also provided. The additional composition may be administered in between doses of the base compositions, or may be provided on a separate and independent periodic basis. The addition compositions generally comprise any combination of less than three of the following:
i) one or more edible or medical mushrooms or an extract, fraction, or isolate thereof;
ii) one or more cannabinoids;
iii) one or more curcuminoids;
iv) one or more terpenes; or
v) one or more flavonoids;

The additional compositions further optionally comprise any combination of one or more of S-adenosylmethionine, methylfolate, omega-3 fatty acids, or a B vitamin, vitamin D, or a compound providing a biologically-available form thereof. In other embodiments, fish oil, primrose oil, or an extract of one or more of black cumin, ginger, fenugreek, licorice, coriander, tomato, carrot, sweet potato, broccoli, green tea, rosemary, hazelnut, walnut, wheat germ, or date, or any combination thereof can also be included. Also suitable for use herein are garlic extract, alpha lipoic acid (ALA), niacin, and nicotinamide, or any combination thereof.

In various embodiments of the methods:
i) the one or more edible or medical mushrooms comprise Agaricus, Auricularia, Clitocybe, Ganoderma, Grifola, Hericium, Lentinus, Leucopaxillus, Phellinus, Pleurotus, Sarcodona, Trametes, Albatrellus, Antrodia, Calvatia, Cordyceps, Flammulina, Fomes, Funlia, Inocybe, Inonotus, Lactarius, Russula, Schizophyllum, Suillus, or Xerocomus;
ii) the cannabinoids comprise one or more of cannabidiol (CBD), cannabinol (CBN), cannabigerol (CBG), or tetrahydrocannabinol (THC);
iii) the curcuminoids comprise one or more of curcumin, demethoxycurcumin (DMC), bisdemethoxycurcumin (BDMC), turmerones, turmeric oil, or a natural or synthetic metabolites, conjugates, or derivatives thereof;
iv) the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene; and
v) the one or more flavonoids comprise chalcones, flavones, isoflavonoids, flavanones, anthoxanthins, anthocyanins, flavonols, or glucosides or other biologically active derivatives or analogs thereof.

The inflammatory disease process can comprise any inflammatory disease process such as any form of chronic inflammation, asthma, chronic peptic ulcer, tuberculosis, periodontitis, ulcerative colitis and Crohn's disease, sinusitis, active hepatitis, or any kind of autoimmune or autoinflammatory disease process, including but not limited to Addison disease, celiac disease, irritable bowel disease, psoriasis, dermatomyositis, Graves disease, Hashimoto thyroiditis, multiple sclerosis, myasthenia gravis, pernicious anemia, reactive arthritis, rheumatoid arthritis, Sjogren syndrome, systemic lupus erythematosus, or Type I diabetes.

In one embodiment of the methods, the composition further comprises an extract or fraction from one or more traditional Jamaican medicinal plants other than Cannabis spp. The traditional medicinal plants comprise ackee, aloe vera, avocado, breadflower, ginger, leaf of life, soursop, tamarind, and/or trumpet in certain embodiments.

The skilled artisan will appreciate that the methods are flexible as set forth herein, an aspect which is particularly useful given the varied and nature of the psychological disorders which they are intended to treat.

In yet another aspect of the disclosure, methods of optimizing a composition for use in treatment of a subject suffering from inflammatory disease process using artificial intelligence are provided herein. The methods generally comprise, for each inflammatory disease process of interest, or for a subject in need of therapeutic compositions for such inflammatory disease process:
a) providing data on the therapeutic effect on the inflammatory disease process of each of:
   i) a plurality of edible or medicinal mushrooms, mushroom extracts, or components thereof;
   ii) a plurality of cannabinoids or combinations thereof;
   iii) a plurality of curcuminoids or combinations thereof;
   iv) a plurality of terpenes or combinations thereof;
   v) a plurality of flavonoids or combinations thereof; and optionally,
   vi) a plurality of combinations of compositions comprising one or more of mushrooms or extracts thereof, cannabinoids, curcuminoids, terpenes, and flavonoid; or
   vii) a plurality of optional ingredients comprising S-adenosylmethionine, methylfolate, omega-3 fatty acids, or a B vitamin or vitamin D or a compound providing a biologically-available form thereof, or combinations thereof, or fish oil, primrose oil, or an extract of one or more of black cumin, ginger, fenugreek, licorice, coriander, tomato, carrot, sweet potato, broccoli, green tea, rosemary, hazelnut, walnut, wheat germ, date, or any combination thereof, garlic extract, alpha lipoic acid (ALA), niacin, or nicotinamide;
b) using an artificial intelligence algorithm to analyze the data for the mushrooms or extracts, cannabinoids, curcuminoids, terpenes, and flavonoids; and
c) generating one or more base profiles of compositions optimized for therapeutic treatment of the inflammatory disease process;
d) optionally, using the artificial intelligence algorithm to analyze the data for the combination compositions and the optional ingredients, and
e) generating one or more complete profiles of compositions with and without the optional ingredients.

In certain embodiments, the inflammatory disease process comprises Achalasia, Addison's disease, Adult Still's disease, agammaglobulinemia, Alopecia areata, amyloidosis, Ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, colitis, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, gout, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), Hidradenitis Suppurativa (HS) (Acne Inversa), hypogammalglobulinemia, IgA Nephropathy, IgG4-related sclerosing disease, immune thrombocytopenic purpura (ITP), inclusion body myositis (IBM), inflammatory bowel disease, interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, Multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, Pemphigus, peripheral neuropathy, perivenous encephalomyelitis, pernicious anemia (PA), POEMS syndrome, polyarteritis nodosa, polyglandular syndromes type I, II, III, Polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, Pure red cell aplasia (PRCA), Pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, Stiff person syndrome (SPS), subacute bacterial endocarditis (SBE), Susac's syndrome, sympathetic ophthalmia (SO), systemic lupus erythematosus, Takayasu's arteritis, temporal arteritis/Giant cell arteritis, thrombocytopenic purpura (TTP), thyroid eye disease (TED), Tolosa-Hunt syndrome (THS), transverse myelitis, Type 1 diabetes, ulcerative colitis (UC), undifferentiated connective tissue disease (UCTD), uveitis, vasculitis, vitiligo, and Vogt-Koyanagi-Harada Disease. Long term disease processes that are associated with inflammation include asthma, chronic peptic ulcer, tuberculosis, periodontitis, ulcerative colitis, Crohn's disease, sinusitis, active hepatitis, Behçet's disease, Chronic Atypical Neutrophilic Dermatosis With Lipodystrophy and Elevated Temperature (CANDLE), Deficiency of the Interleukin-1 Receptor Antagonist (DIRA), Familial Mediterranean Fever (FMF), Neonatal Onset Multisystem Inflammatory Disease (NOMID), Tumor Necrosis Factor Receptor-Associated Periodic Syndrome (TRAPS), or other autoimmune or autoinflammatory disease. In certain embodiments, chronic inflammatory conditions are treated, or conditions that are associated with chronic inflammation such as heart disease or Alzheimer's disease. Generally, the data for the method are obtained from original experiments and/or reviews of the relevant scientific literature.

The artificial intelligence algorithm can comprise any useful software or algorithm approach capable of making the distinctions required. In various embodiments, the algorithm comprises a classification algorithm, a regression algorithm, a clustering algorithm, or a combination thereof.

In one embodiment, the methods comprise a classification algorithm that is a naïve Bayes algorithm, decision tree, random forest algorithm, Support Vector Machines, or K Nearest Neighbor algorithm.

In another embodiment, the methods comprise a regression algorithm that is a liner regression, lasso regression, logistic regression, or multivariate regression.

In yet another embodiment, the methods comprise a clustering algorithm that is a K-means clustering, fuzzy C-means algorithm, expectation-maximization algorithm, or hierarchical clustering algorithm.

The skilled artisan will appreciate that the methods are designed to optimize the compositions, and that such optimization as set forth above can be with respect to each particular inflammatory disease process or even each particular variant of such a condition. However, the compositions can also be optimized for, and a profile of relevant compositions generated for each particular subject, e.g. for a ‘personalized medicine’ approach.

Thus, also provided herein are the methods comprising the additional step of providing subject-specific data comprising, e.g. initial or subsequent blood work, enzyme test results, bioinformatic data (including measurements of e.g. the genome, transcriptome, proteome, metabolome, or any portion thereof, for a subject), specific symptomology, or the like. The artificial intelligence algorithm is then used to further optimize the composition based on those data in addition to the disorder-specific data. The optimized formulation of the compositions may also be changed based on data from the results from an initial treatment, subsequent treatment, or based on subsequent tests of the subject.

In certain embodiments, the inflammatory disease process comprises any form of chronic inflammation, asthma, chronic peptic ulcer, tuberculosis, periodontitis, ulcerative colitis and Crohn's disease, sinusitis, active hepatitis, or any kind of autoimmune or autoinflammatory disease process, including but not limited to Addison disease, celiac disease, irritable bowel disease, psoriasis, dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, or Type I diabetes.

The plurality of edible or medicinal mushrooms, mushroom extracts, or components for which data are analyzed generally comprise one or more of the species Agaricus blazei, Albatrellus confluens, Antrodia camphorate, Boletus badius, Clitocybe maxima, Cordyceps militaris, Cordyceps sinensis, Cordyceps liangshanensis, Cordyceps gunnii, Cordyceps cicadicola, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, or Trametes versicolor.

A further aspect of the invention provides methods for the treatment of inflammatory disease process including a chronic inflammatory disease condition, or a disease, such as heart disease or Alzheimer's disease, that while itself is not an inflammatory disease, is characterized by chronic inflammation. The methods generally comprise the step of administering to a patient in need thereof a composition comprising at least one edible or medicinal mushroom or extract thereof, in combination with at least one cannabinoid, at least one curcuminoid, at least one terpene, and at least one flavonoid. In one embodiment the at least one cannabinoid, at least one curcuminoid, at least one terpene, and at least one flavonoid are administered separately from, sequentially to, or simultaneously with the edible or medicinal mushroom or extract thereof. In another embodiment, the at least one edible or medicinal mushroom or extract thereof is also administered separately from, sequentially to, or simultaneously with the cannabinoid, curcuminoid, terpene, and flavonoid.

Again, the composition with respect to this aspect of the disclosure can comprise any of the compositions described hereinabove. In one embodiment of the methods:
i) the at least one edible or medicinal mushroom or extract comprises comprise as Agaricus blazei, Albatrellus confluens, Antrodia camphorate, Boletus badius, Clitocybe maxima, Cordyceps militaris, Cordyceps sinensis, Cordyceps liangshanensis, Cordyceps gunnii, Cordyceps cicadicola, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, or Trametes versicolor;
ii) the at least one cannabinoid comprises CBD, CBG, CBN, and THC extracted from Cannabis sativa Ringo's Gift strain;
iii) the at least one curcuminoid comprises one or more of curcumin, demethoxycurcumin (DMC), bisdemethoxycurcumin (BDMC), turmerones, turmeric oil, or a natural or synthetic metabolite, conjugate, or derivative thereof;
iv) the at least one terpene comprises limonene, myrcene, beta-caryophyllene, linalool, alpha pinene, or a combination thereof; and
v) the at least one flavonoid comprises a chalcone, flavone, isoflavonoid, flavanone, anthoxanthin, anthocyanin, flavonol, or glucoside or other biologically active derivatives or analogs thereof.

In various embodiments of the methods, one or more of the components have one more of the following functions:
a) induces apoptosis of cells involved in maintaining the inflammatory disease process;
b) disrupts one or more inflammatory cellular responses from proinflammatory cells macrophages;
c) blocks activity of protein kinase C theta (PKCθ)
d) inhibits or alters the creation of new adipose tissue;
e) disrupts the functioning of one or more cellular mediators of inflammation comprising at least one interleukin, tumor necrosis factor; nuclear factor, intercellular adhesion molecule, inducible type cyclooxygenase, prostaglandin, lipoxygenase, or nitric oxide synthase; or
f) inhibits an inflammation-related disturbance in the blood-brain barrier.

In yet another embodiment of the methods provided in this aspect of the disclosure, the composition further comprises an extract or fraction from one or more traditional Jamaican medicinal plants other than Cannabis spp. Traditional medicinal plants such as ackee, aloe vera, avocado, breadflower, ginger, leaf of life, soursop, tamarind, and trumpet are contemplated as useful herein.

The scope of the invention is set forth in the claims appended hereto, subject, for example, to the limits of language. Although specific terms are employed to describe the invention, those terms are used in a generic and descriptive sense and not for purposes of limitation. Moreover, while certain presently preferred embodiments of the claimed invention have been described herein, those skilled in the art will appreciate that such embodiments are provided by way of example only. In view of the teachings provided herein, certain variations, modifications, and substitutions will occur to those skilled in the art. It is therefore to be understood that the invention may be practiced otherwise than as specifically described, and such ways of practicing the invention are either within the scope of the claims, or equivalent to that which is claimed, and do not depart from the scope and spirit of the invention as claimed.

1. A composition comprising one or more edible or medicinal mushrooms or an extract, fraction, or isolate thereof, one or more cannabinoids, one or more terpenes, one or more curcuminoids, and optionally, one or more flavonoids.

2. The composition of claim 1 wherein the edible or medicinal mushroom comprises one or more of Agaricus, Agrocybe, Albatrellus, Amanita, Boletus, Cantharellus, Cordyceps, Caripia, Cyathus, Daldinia, Dictyophora, Elaphomyces, Flammulina, Fomitopsis, Grifola, Ganoderma, Geastrum, Inonotus, Lactarius, Lentinus, Lyophyllum, Phellinus, Pholiota, Pleurotus, Poria, and Termitomyces.

3. (canceled)

4. The composition of claim 1 wherein the edible or medicinal mushroom comprises fresh mushroom, dried mushroom, lyophilized mushroom, a mushroom powder, an aqueous or ethanolic extract of mushroom, or concentrate thereof, or a partially purified or purified mushroom fraction or component, or an isolate thereof.

5. The composition of claim 4 wherein the aqueous or ethanolic extract of mushroom, or concentrate thereof, or the partially purified or purified mushroom fraction or component, or isolate thereof are enriched for one or more of phenolic acids, flavonoids, triterpenes, carotenoids, stilbenes, sterols, fatty acids, lignans, chitosan, polyphenols, polysaccharides, glycosides, glucans, polysaccharide-protein complexes, glycoproteins, polysaccharopeptide, krestin, tocopherols, peptides, cyclic peptides, or amino compounds found in the mushroom.

6. The composition of claim 1 wherein the curcuminoid comprises one or more of curcumin, demethoxycurcumin (DMC), bisdemethoxycurcumin (BDMC), turmerones, turmeric oil, or a natural or synthetic metabolite, conjugate, or derivative thereof.

7. (canceled)

8. The composition of claim 1 wherein the cannabinoids comprise one or more of cannabidiol (CBD), cannabinol (CBN), cannabigerol (CBG), or tetrahydrocannabinol (THC).

9. The composition of claim 8 wherein the THC is present at less than about 0.3 percent, or wherein the ratio of CBD to THC is about 1:5 to about 30:1 or more.

10. (canceled)

11. The composition of claim 1 wherein the cannabinoids are derived from Cannabis spp. or an extract thereof.

12. (canceled)

13. (canceled)

14. (canceled).

15. The composition of claim 11 wherein the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, phellandrene, pinene, phytol, pulegone, terpinene, terpineol, terpinolene, or valencene.

16. The composition of claim 15 wherein the terpenes are derived from Cannabis spp. or an extract thereof.

17. (canceled)

18. The composition of claim 16 wherein the Cannabis spp. is a Cannabis sativa, which for the cannabinoids and the terpenes independently comprises one or more of Ringo's Gift, Harle Tsu, ACDC, Charlotte's Web, The Gift, or Pineberry strains.

19. (canceled)

20. The composition of claim 1 wherein the one or more flavonoids comprise chalcones, flavones, isoflavonoids, flavanones, anthoxanthins, anthocyanins, flavonols, or glucosides or other biologically active derivatives or analogs thereof.

21. (canceled)

22. (canceled)

23. The composition of claim 1 further comprising an extract or fraction from one or more traditional Jamaican medicinal plants other than Cannabis spp.

24. (canceled)

25. (canceled)

26. A method of treating a subject suffering from an inflammatory disease process which comprises administering to the subject a therapeutically effective dose of a composition comprising one or more edible or medicinal mushrooms or an extract, fraction, or isolate thereof, one or more cannabinoids, one or more curcurminoids, one or more terpenes, and optionally, one or more flavonoids;
wherein the inflammatory disease process comprises Achalasia, Addison's disease, Adult Still's disease, agammaglobulinemia, Alopecia areata, amyloidosis, Ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Balli disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, colitis, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, gout, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), Hidradenitis Suppurativa (HS) (Acne lnversa), hypogammalglobulinemia, IgA Nephropathy, IgG4-related sclerosing disease, immune thrombocytopenic purpura (ITP), inclusion body myositis (IBM), inflammatory bowel disease, interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, Multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, Pemphigus, peripheral neuropathy, perivenous encephalomyelitis, pernicious anemia (PA), POEMS syndrome, polyarteritis nodosa, polyglandular syndromes type I, II, III, Polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, Pure red cell aplasia (PRCA), Pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjogren's syndrome, sperm & testicular autoimmunity, Stiff person syndrome (SPS), subacute bacterial endocarditis (SBE), Susac's syndrome, sympathetic ophthalmia (SO), systemic lupus erythematosus, Takayasu's arteritis, temporal arteritis/Giant cell arteritis, thrombocytopenic purpura (TTP), thyroid eye disease (TED), Tolosa-Hunt syndrome (THS), transverse myelitis, Type 1 diabetes, ulcerative colitis (UC), undifferentiated connective tissue disease (UCTD), uveitis, vasculitis, vitiligo, and Vogt-Koyanagi-Harada Disease. Long term disease processes that are associated with inflammation include asthma, chronic peptic ulcer, tuberculosis, periodontitis, ulcerative colitis, Crohn's disease, sinusitis, active hepatitis, Behçet's disease, Chronic Atypical Neutrophilic Dermatosis With Lipodystrophy and Elevated Temperature (CANDLE), Deficiency of the Interleukin-I Receptor Antagonist (DIRA), Familial Mediterranean Fever (FMF), Neonatal Onset Multisystem Inflammatory Disease (NOMID), Tumor Necrosis Factor Receptor-Associated Periodic Syndrome (TRAPS), or other autoimmune or autoinflammatory disease.

27. The method of claim 26 wherein the composition further comprises one or more optional ingredients comprising S-adenosylmethionine, methylfolate, omega-3 fatty acids, or a B vitamin, vitamin D or a compound providing a biologically-available form thereof, fish oil, primrose oil, or an extract of one or more of black cumin, ginger, fenugreek, licorice, coriander, tomato, carrot, sweet potato, broccoli, green tea, rosemary, hazelnut, walnut, wheat germ, date, or combinations thereof, garlic extract, alpha lipoic acid (ALA), niacin, or nicotinamide.

28. The method of claim 26 wherein the edible or medicinal mushroom comprises one or more of Agaricus, Agrocybe, Albatrellus, Amanita, Boletus, Cantharellus, Cordyceps, Caripia, Cyathus, Daldinia, Dictyophora, Elaphomyces, Flammulina, Fomitopsis, Grifola, Ganoderma, Geastrum, Inonotus, Lactarius, Lentinus, Lyophyllum, Phellinus, Pholiota, Pleurotus, Poria, and Termitomyces.

29. The method of claim 26 wherein the cannabinoids comprise one or more of cannabidiol (CBD), cannabinol (CBN), cannabigerol (CBG), or tetrahydrocannabinol (THC) wherein the THC is present at less than about 0.3 percent or wherein the ratio of CBD to THC is about 1:5 to about 30:1 or more.

30. (canceled)

31. (canceled)

32. (canceled)

33. The method of claim 29 wherein the cannabinoids and the terpenes are independently derived from one or more of Ringo's Gift, Harle Tsu, ACDC, Charlotte's Web, The Gift, or Pineberry strains of Cannabis sativa.

34. (canceled)

35. (canceled)

36. (canceled)

37. (canceled)

38. (canceled)

39. (canceled)

40. (canceled)

41. (canceled)

42. (canceled)

43. The method of claim 26 further comprising an additional step of providing to the subject an additional composition comprising any combination of less than three of the following:
a) one or more edible or medical mushrooms or an extract, fraction, or isolate thereof;
b) one or more curcuminoids;
c) one or more cannabinoids;
d) one or more terpenes; or
e) one or more flavonoids;
and optionally, any combination of one or more of S-adenosylmethionine, methylfolate, omega-3 fatty acids, or a B vitamin, vitamin D, or a compound providing a biologically-available form thereof, fish oil, primrose oil, or an extract of one or more of black cumin, ginger, fenugreek, liquorice licorice, coriander, tomato, carrot, sweet potato, broccoli, green tea, rosemary, hazelnut, walnut, wheat germ, date, or combinations thereof, garlic extract, alpha lipoic acid (ALA), niacin, or nicotinamide wherein:
f) the one or more edible or medical mushrooms comprise Agaricus, Agrocybe, Albatrellus, Amanita, Boletus, Cantharellus, Cordyceps, Caripia, Cyathus, Daldinia, Dictyophora, Elaphomyces, Flammulina, Fomitopsis, Grifola, Ganoderma, Geastrum, Inonotus, Lactarius, Lentinus, Lyophyllum, Phellinus, Pholiota, Pleurotus, Poria, and Termitomyces;
g) the cannabinoids comprise one or more of cannabidiol (CBD), cannabinol (CBN), cannabigerol (CBG), or tetrahydrocannabinol (THC);
h) the curcuminoids comprise one or more of curcumin, demethoxycurcumin (DMC), bisdemethoxycurcumin (BDMC), turmerones, turmeric oil, or a natural or synthetic metabolite, conjugate, or derivative thereof;
i) the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene; and
j) the one or more flavonoids comprise chalcones, flavones, isoflavonoids, flavanones, anthoxanthins, anthocyanins, flavonols, or glucosides or other biologically active derivatives or analogs thereof.

44. (canceled)

45. (canceled)

46. (canceled)

47. (canceled)

48. (canceled)

49. (canceled)

50. (canceled)

51. (canceled)

52. A method for the treatment of inflammatory disease process comprising the step of administering to a patient in need thereof a composition comprising at least one edible or medicinal mushroom or extract thereof, in combination with at least one cannabinoid, at least one curcuminoid, at least one terpene, and at least one flavonoid; wherein the at least one cannabinoid, at least one terpene, and at least one flavonoid is administered separately from, sequentially to, or simultaneously with the edible or medicinal mushroom; and wherein the at least one edible or medicinal mushroom or extract thereof is also administered separately from, sequentially to, or simultaneously with the cannabinoid, terpene, and flavonoid.
